(19) United States
(12) Patent Application Publication
(10) Pub. No.: US 2022/0354821 A1
(43) Pub. Date: Nov. 10, 2022

(54) DEFINED DOSE CANNABIS POD

(71) Applicant: Emerald Health Therapeutics Canada Inc., Vancouver (CA)

(72) Inventors: Christopher WAGNER, Vancouver (CA); Nancy HARRISON, Vancouver (CA)

(21) Appl. No.: 17/586,618

(22) Filed: Jan. 27, 2022

Related U.S. Application Data
(63) Continuation of application No. 16/981,211, filed on Sep. 15, 2020, now abandoned.
(60) Provisional application No. 62/645,070, filed on Mar. 19, 2018.

(51) Int. Cl. A61K 31/352; A61J 1/03; A61K 9/48; A61K 31/05

(52) U.S. Cl. CPC A61K 31/352; A61J 1/035; A61K 9/4816; A61K 31/05

(57) ABSTRACT

This invention relates to novel products of Cannabis flower for medicinal and/or recreational use, and for methods of making such products.

### CROSS-REFERENCE

This application is a continuation of U.S. patent application Ser. No. 16/981,211, filed Sep. 15, 2020, which is the U.S. National Stage Application under 35 U.S.C. § 371 of International Application No. PCT/IB2019/000278, filed Mar. 18, 2019, which claims the benefit of U.S. Provisional Application No. 62/645,070, filed Mar. 19, 2018, each of which is hereby incorporated by reference in its entirety

### BACKGROUND OF THE INVENTION

One bourbon, one scotch and one beer all have roughly the same amount of alcohol in them based on the volume they contain. Users recognize that the mildly intoxicating effects of such drinks are consistent and predictable, despite the wide variety of brands, qualities and prices.

But no such equivalency exists for Cannabis. Inspection of a Cannabis plant product by eyesight, taste or smell provides no reliable guidance as to the amount or potency of physiologically and/or psychotropically active cannabinoids in the product. This presents the user with risks and uncertainty that can only be effectively resolved by consuming small amounts of the sample, waiting for an effect, and then consuming another small part of the sample

### SUMMARY OF THE INVENTION

The present disclosure relates to encapsulated Cannabis pods which contain processed plant source material and which allow the cannabinoid ingredients to be released through vaporization. The Cannabis pods provide reliable and consistent defined doses of selected cannabinoids for recreational and/or medicinal users. The Cannabis pods may mitigate one or more hazards associated with the use of marijuana, particularly concerns regarding consistency of dosing. The present invention provides a family of encapsulated pod products and methods for preparation thereof.

Some embodiments provide Cannabis pods comprising a pre-defined total amount of one or more active ingredients in ranges of about 0 mg-100 mg. Active ingredients may be selected from the group including THC, THCA, CBD and CBDA.

Some embodiments provide Cannabis pods wherein the active ingredients may be present in predetermined ratios to achieve for example, a desired effect, or for a particular purpose.

In some embodiments, the pods comprise low THC:THCA ratios. Low THC:THCA pods may comprise THC in ranges of about 0 mg-1 mg and THCA in ranges of about 9 mg to 90 mg. Low THC:THCA pods may further comprise CBD in ranges of about 7 mg-75 mg.

In some embodiments, the pods comprise low THCA:THC ratios. Low THCA:THC pods may comprise THCA in ranges of about 0 mg-1 mg and THC in ranges of about 9 mg-90 mg. Low THCA:THC pods may further comprise CBD in ranges of about 7 mg-75 mg.

In some embodiments, the pods are substantially free of THC-type compounds. Pods substantially free of THC-type compounds may comprise CBD in ranges of about 7 mg-75 mg, THC in ranges of about 0 mg-1 mg, THCA in ranges of about 0 mg-1 mg.

In some embodiments, the pods described herein may be for use for direct vaporization.

In some embodiments, the invention provides methods of producing Cannabis pods described herein. The methods comprising a) a preparation step; and b) an encapsulation step.

The pods described herein can preferably be pressed into standardized amounts through compaction. These pods provide a defined dose of selected cannabinoids and preferably are associated with a signifier which identifies such defined dose for the consumer. The pods can be packaged individually in blister pack or in a multi-pod pack.

Described herein are methods of preparing a Cannabis pod comprising a desired defined dose of one or more desired cannabinoids, the method comprising: (a) preparing or obtaining a ground or milled Cannabis plant material preparation; (b) passing the Cannabis plant material preparation through a mesh or sieve to obtain a retained fraction of the material which is retained in the mesh or sieve and a pass-through fraction of the material which has passed through the mesh or sieve; (c) determining the content of the one or more cannabinoids of interest in the retained fraction and/or the pass-through fraction; (d) based on the content determined in step (c), determining a target amount of retained fraction material, pass-through material, or a combination thereof, to be incorporated into the Cannabis pod such that the pod includes the desired defined dose of the one or more desired cannabinoids; and (e) encapsulating the target amount within a textile, preferably of primarily natural fiber, said textile having a basis weight of 12-28 g/m² permitting air flow at or below minimal inspiratory resistance, thereby forming the Cannabis pod. In methods described herein, the target amount contains about 0.1 to about 1.0 grams total mass of Cannabis material and further comprises: 0.1-100 mg tetrahydrocannabinolic acid (THCA), 0.1-100 mg tetrahydrocannabinol (THC), 0.1-100 mg cannabidiolic acid (CBDA), and/or 1-100 mg cannabidiol (CBD). In methods described herein, the pod comprises THCA in an amount between 1-5 mg or between 5-165 mg; THC in an amount less than 1.0 mg; and CBDA in an amount between 0.1-70 mg, and has a total mass of 100-500 mg. In methods described herein, the pod comprises THCA in an amount less than 5.0 mg; THC in an amount between 1-5 mg or between 5-135 mg; and CBD in an amount between 0.1-70 mg, and has a total mass of 100-500 mg. In methods described herein, the pod comprises THCA in an amount less than 1.0 mg; THC in an amount less than 1.0 mg; and CBD in an amount between 5-90 mg, and has a total mass of 100-500 mg. In methods described herein, the Cannabis plant material in the preparation of a) has not been previously exposed to accelerated dehydration at greater than about 100° C. In methods described herein, following step a), the preparation is heated at above about 105° C. but below about 150° C. for a duration of about 5 minutes to about 30 minutes. In methods described herein, prior to step a), the Cannabis preparation has been heated at above about 105° C. but below about 150° C. for a duration of about 5 minutes to about 30 minutes. In methods described herein, further comprising after step b), packaging the pod individually in a blister pack impermeable to gas exchange. In methods described herein, further comprising after step c), packaging the pod in a re-sealable multi-pod package impermeable to gas exchange. In methods described herein, the Cannabis plant material is derived from one or more members of a species selected from the group consisting of Cannabis sativa, Cannabis indica, Cannabis ruderalis, and hybrids thereof. In methods described herein, the Cannabis is Cannabis indica. In methods described herein, the Cannabis plant material preparation is from the variety Charlotte's Web. In methods described herein, the Cannabis plant material preparation is prepared from Cannabis inflorescence. In methods described herein, the preparation of step a) further comprises one or more additives selected from among terpenes, terpenoids, pod stabilizers, humectants, vaporization aids, fillers and flavours. In methods described herein, the Cannabis plant material preparation in a) has been previously ground to sieve through a mesh of not larger than 0.595 mm in any surface dimension.

Described herein is a defined dose Cannabis pod comprising a defined dose of: (a) 0.1-100 mg tetrahydrocannabinolic acid (THCA), (b) 0.1-100 mg tetrahydrocannabinol THC, (c) 0.1-100 mg cannabidiolic acid CBDA; and/or (d) 0.1-100 mg cannabidiol CBD in a ground Cannabis plant material preparation contained within a textile of primarily natural fiber, said textile having a basis weight of 12-28 g/m² and permitting air flow at or below minimal inspiratory resistance. Further described herein is a composition, wherein the Cannabis plant material preparation includes material derived from one or more Cannabis plants from a species selected from the group consisting of Cannabis sativa, Cannabis indica, Cannabis ruderalis, and hybrids thereof. Further described herein is a composition, wherein the Cannabis species is Cannabis indica. Further described herein is a composition, wherein the Cannabis plant material preparation includes material prepared from Cannabis inflorescence. Further described herein is a composition comprising one or more additives selected from among terpenes, terpenoids, pod stabilizers, humectants, vaporization aids, fillers and flavours. Further described herein is a composition, wherein the amount of THC is less than a psychotropic dose. Further described herein is a composition, wherein the amount of THCA is less than 1.0 mg. Further described herein is a composition, comprising: THCA in an amount between 1-5-165 mg, THC in an amount less than 1.0 mg; and CBDA in an amount between 0.1-70 mg, and having total mass 100-500 mg. Further described herein is a composition comprising: THCA in an amount less than 5.0 mg, THC in an amount between 1-5 mg or between 5-135 mg; and CBD in an amount between 0.1-70 mg, and having total mass 100-500 mg. Further described herein is a composition comprising: THCA in an amount less than 1.0 mg, THC in an amount less than 1.0 mg; and CBD in an amount between 5-90 mg, and having total mass 100-500 mg. Further described herein is a composition, wherein one or more of the Cannabis plant material preparation is prepared from one or more Cannabis plants selected from the group consisting of Charlotte's Web and other high CBDA, low THCA plant varieties. Further described herein is a composition, wherein the textile contains no synthetic polymer. Further described herein is a composition, wherein the textile is organically sourced. Further described herein is a composition, wherein the pod comprises no chemical adhesive. Further described herein is a composition sealed individually in a blister pack impermeable to gas exchange, and optionally in an inert gas atmosphere. Further described herein is a composition in a re-sealable multi-pod package impermeable to gas exchange. Further described herein is a composition, associated with a signifier which provides an observer with information on the defined dose of selected cannabinoids in the composition. Further described herein is a composition, wherein the packaging is associated with a signifier which provides an observer with information on the defined dose of selected cannabinoids in the composition. Further described herein is a composition, wherein the pod comprises a pull-tab disposed to release the pod contents at the user's option, said pull-tab affixed to a stiffening structure embossed in or underlying said textile and arranged in a pattern such that pulling of the pull-tab by a user tears the textile along a creased or weakened folding line that defines the intended tearing line across the textile. Further described herein is a composition, wherein the stiffening structure comprises embossed, corrugated, creased, folded or heat-shaped material. Further described herein is a composition, wherein the intended tearing line extends in a direction substantially parallel to the long axis of the pod. Further described herein is a composition, wherein a plurality of elongated stiffening elements extends from the pull-tab towards outer edges of the pod. Further described herein is a composition, wherein the pull-tab includes a concave groove dimensioned to aid in grasping thereof. Further described herein is a composition associated with signifier which provides an observer with information on the defined dose of THC in the composition. Further described herein is a composition associated with a signifier which provides an observer with information on the defined dose of cannabinoids in the composition, wherein such cannabinoids are selected from among THCA, THC, CBDA, and CBD.

Described herein are methods of preparing a defined dose Cannabis pod comprising a defined dose of: (a) preparing a Cannabis preparation of about 0.1 to about 1.0 grams total mass of Cannabis plant material, comprising: 0.1-100 mg tetrahydrocannabinolic acid (THCA), 0.1-100 mg tetrahydrocannabinol (THC), 0.1-100 mg cannabidiolic acid (CBDA), and/or 1-100 mg cannabidiol (CBD); and (b) encapsulating the Cannabis preparation within a textile of primarily natural fiber, said textile having a basis weight of 12-28 g/m² permitting air flow at or below minimal inspiratory resistance. Further described herein are methods, wherein the pod comprises: THCA in an amount between 5-165 mg; THC in an amount less than 1.0 mg; and; CBDA in an amount between 0.1-70 mg, and has a total mass of 100-500 mg. Further described herein are methods, wherein the pod comprises: THCA in an amount less than 5.0 mg; THC in an amount between 1-5 mg or between 5-135 mg; and CBD in an amount between 0.1-70 mg, and has a total mass of 100-500 mg. Further described herein are methods, wherein the pod comprises: THCA in an amount less than 1.0 mg; THC in an amount less than 1.0 mg; and CBD in an amount between 5-90 mg, and has a total mass of 100-500 mg. Further described herein are methods, wherein the Cannabis plant material in the preparation of a) has not been previously exposed to accelerated dehydration at greater than about 100° C. Further described herein are methods, wherein following step b), the preparation is heated at above about 105° C. but below about 150° C. for a duration of about 5 minutes to about 30 minutes. Further described herein are methods, wherein prior to step a), the Cannabis preparation has been heated at above about 105° C. but below about 150° C. for a duration of about 5 minutes to about 30 minutes. Further described herein are methods, further comprising after step b), packaging the pod individually in a blister pack impermeable to gas exchange. Further described herein are methods, further comprising after step b), packaging the pod in a re-sealable multi-pod package impermeable to gas exchange. Further described herein are methods, wherein the Cannabis plant material is derived from one or more Cannabis plants that are a member of a species selected from the group consisting of Cannabis sativa, Cannabis indica, Cannabis ruderalis, and hybrids thereof. Further described herein are methods, wherein the Cannabis is Cannabis indica. Further described herein are methods, wherein the Cannabis plant material is derived from the variety Charlotte's Web. Further described herein are methods, wherein the Cannabis plant material is prepared from Cannabis inflorescence. Further described herein are methods, wherein the preparation of step a) further comprises one or more additives selected from among terpenes, terpenoids, pod stabilizers, humectants, vaporization aids, fillers and flavours. Further described herein are methods, wherein the Cannabis preparation in a) has been previously ground to sieve through a mesh of not larger than 0.595 mm in any surface dimension.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Method of forming a defined dose Cannabis pod.

### DETAILED DESCRIPTION OF THE INVENTION

Unpredictability is a hallmark of Cannabis. It starts with the species. The most common varieties, also referred to as chemical varieties or chemovars, worldwide, Cannabis sativa, Cannabis indica and Cannabis ruderalis, have distinct but overlapping ranges of cannabinoids. Over 100 cannabinoids may be found in these plants. Varieties and strains are continually crossed and or hybridized, generating different cannabinoid ratios. Further, the cannabinoid ratios within a single variety can be influenced by the conditions of cultivation, especially light cycle, temperature, soil condition, nutrient availability, and pathogen exposure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Harvesting and processing of Cannabis provides further room for variation. Every farmer knows the challenge of deciding when a crop is ready for harvest. Timing of harvest will influence cannabinoid amounts and ratios within a single variety. The degree of drying and/or curing the harvest will further influence the amount of cannabinoids by weight. Uncertainty also enters the process because the final product may be prepared exclusively from the inflorescence (also called flower or bud, being the plant part containing the highest cannabinoid concentrations), or alternatively some producers may feed other plant parts such as leaves and stem back into the final product as fillers.

The result is that a Cannabis product presented to a consumer can have, by weight, anywhere from 0% up to greater than 30% of selected cannabinoids, and the ratios between individual cannabinoids can be extraordinarily diverse.

Significantly, consumers cannot tell by visual inspection of a Cannabis preparation what amounts of the primary physiologically active cannabinoids tetrahydrocannabinol (THC), cannabidiol (CBD) and cannabigerol (CBG) are present. This rather critical uncertainty results from the fact that during the cultivation phase, Cannabis naturally synthesizes only the low potency precursors Tetrahydrocannabinolic acid (THCA), cannabidiolic acid CBDA and cannabigerolic acid (CBGA). These compounds convert respectively to THC (the primary psychoactive cannabinoid), and CBD and CBG (both significant non-psychoactive analgesic and anti-inflammatory cannabinoids) via decarboxylation. Decarboxylation may be induced by heating over 105° C. and/or by exposure to ultraviolet (UV) light. Whether the product has been so treated is not immediately apparent to a consumer. Among other things, this uncertainty creates a safety issue, because if the product is accidentally orally consumed by children or pets, there is no way of knowing if an alarming psychotropic event will result. (Gastric acids do not convert THCA to THC). See Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271.

Another area of relevant background pertains to two main methods of consuming Cannabis: Smoking and vaping. Smoking is achieved with a wide variety of combustion devices, including cigarettes. The auto-ignition temperature of dried Cannabis is approximately 232° C. Ignition leads to much higher temperatures, all of which are sufficient to decarboxylate cannabinoids, which are then inhaled. “Vaping” is a method of vaporization whereby Cannabis is heated to a point below the auto-ignition point but above the THCA decarboxylation point (105° C.) and above the evaporation point of cannabinoids (e.g. approximately 157-226° C.). The user thereby inhales an evaporant that contains cannabinoids but does not contain combustion products.

Concerns about safety and unpredictability of Cannabis have historically been ignored by consumers. With increasing social and legal acceptance of Cannabis use, for example in North American jurisdictions including Canada and California, these concerns are likely to rapidly increase over time. This patent application pertains to Cannabis products with standardized, consistent amounts of cannabinoids so that users can find consistency and predictability which they have grown to expect in the field of alcoholic beverages.

The present invention relates to defined dose pods of encapsulated Cannabis flower that are consumed by consumers particularly by direct vaporization. The pods comprise a defined dose of one or more cannabinoids and are preferably associated with a signifier of the defined dose.

The present invention includes embodiments wherein the defined doses are distinct, and the uses are distinct. For example, embodiments include:
1) Low THC, high THCA Cannabis pod. This embodiment is a safety pod. It will have insignificant psychotropic activity on pets or children if accidentally orally ingested. If orally consumed it will be a non-psychotropic medicinal product. When vaped, the THCA converts to THC and delivers psychotropic effect.
2) Low THCA, high THC Cannabis pod. This pod requires a curing process (treatment at 105-150 C) either of the initial Cannabis preparation, or of the final pod. It may optionally be used in orally consumed products to induce a psychotropic effect.
3) High CBD and negligible THCA or THC Cannabis pod. This pod is made from Cannabis varieties and cultivars which synthesize little or no THCA but abundant CBDA (which converts to CBD upon curing or vaping). Charlotte's Web is a suitable Cannabis cultivar to use in the preparation. This embodiment provides a non-psychotropic medicinal product when vaped.

The invention provides numerous improvements over the art, and may be associated with further alternative improvements which may be used in combination or alone to provide advantages for the pod, including but not limited to:
A step which includes measuring and adjusting each cannabinoid to within +/−5% of its defined dose amount.
Association of the pod or its packaging with a signifier which provides an observer with information on the defined dose of selected cannabinoids in the composition.
Use of preferred primarily natural fiber textiles for encapsulating the Cannabis preparation.
Avoidance of synthetic adhesives during the encapsulation process.
Selection of the Cannabis from among Cannabis sativa, Cannabis indica, Cannabis ruderalis, and hybrids thereof
Inclusion of additives selected from among pod stabilizers, humectants, vaporization aids, fillers and flavours. In some embodiments, the pod(s) include certain terpene or terpenoid compounds. For example, in some embodiments, pods include added limonene, providing a lemon scent to the consumer. In other embodiments, pods include added myrcene. Such pods including added myrcene may be useful as sleep aids.
Pods sealed individually in a blister pack impermeable to gas exchange, optionally in an inert gas environment.
A re-sealable multi-pod package impermeable to gas exchange.

The methods of the invention improve upon the art by providing such steps as:
A precise and controlled method of grinding the Cannabis preparation to sieve through a mesh of not larger than 1.5 mm in any surface dimension.
A step of curing the product by temperature or UV treatment prior to encapsulation or after.

### Definitions

“Cannabis” as used herein includes all members of the Cannabis genus, including without limitation Cannabis sativa, Cannabis indica, Cannabis ruderalis, and hybrids thereof “Cannabis” also includes Charlotte's Web and other high CBD, low THCA plant varieties.

“Cannabis inflorescence” means a cluster of flowers on a branch or a system of branches. An inflorescence is categorized on the basis of the arrangement of flowers on a main axis and by the timing of its flowering. Types of inflorescence may include solitary, spikes, racemes, and panicles. Cannabis is an example of a plant that forms racemes or “buds”. In a raceme, a flower develops at the upper angle (axil) between the stem and branch of each leaf along a long, unbranched axis.

“Cured” means harvested Cannabis which has been heated or cooked above 105° C. but below 115° C. for sufficient duration (30 minutes recommended) to convert essentially all THCA to THC by heat-induced decarboxylation.

“Uncured” means fresh harvest, unprocessed, or processed harvest which has not been exposed to temperature above 105° C. Product exposed to drying or accelerated dehydration which does not exceed 100° C. is considered uncured.

“Defined dose” means the dose of one or more active ingredients (typically cannabinoids) has been selected during the production process and is signified to a consumer by a signifier associated with the object.

“Kief” refers to a composition predominantly containing isolated trichome nodules removed from the Cannabis inflorescence. Trichome nodules arise during the flowering stage on the outer surface of the inflorescence. They are enriched in cannabinoids. Kief may be removed after harvest by gently rubbing flowers (typically dried flowers) together, such as by hand or in a tumbling drum. Kief is captured on a 65-125 micron mesh. Immature trichomes will pass through such a mesh. Larger particles would be unwanted plant material. Grinding processes must consider whether the kief is to be separated or combined in the final grind.

“Minimal inspiratory resistance” of a textile means that the textile by itself permits air flow such that an average human would not have a notable resistance to oral inspiration or inhalation of air if the textile were applied to cover the mouth/nose.

“Pod” means a single unit package containing ground plant source material. The single unit package comprises an outer layer of heat resistant fibrous material that encompasses and encapsulates a ground plant source material therein and is resistant to burning or vaporizing, as appropriate, during use.

“Psychotropic dose” means a dose of THC capable of affecting a user's mental state. Some people begin to notice an effect at doses greater than 1 mg THC. “Less than a psychotropic dose” of THC means less than 1 mg THC.

“Pull-tab” means a tab or ring structure that is pulled to open a container. The pull-tab may include a concave groove dimensioned to aid in grasping thereof.

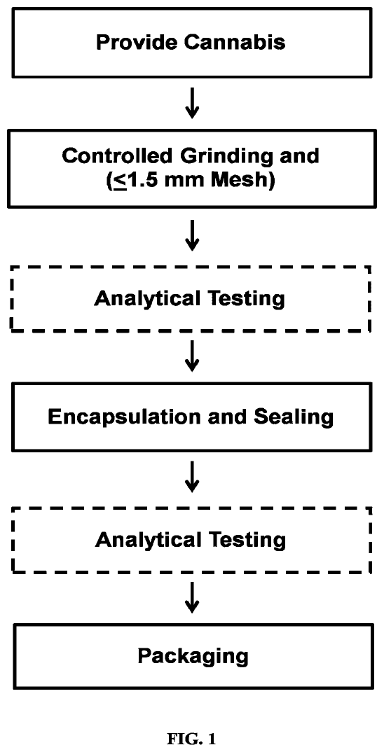

The methods of the invention which may be used to provide a composition of the invention will now be set out stepwise. The method steps and selected optional embodiments are generally set out in FIG. 1.

### Grinding the Cannabis Preparation

The method of the invention requires a Cannabis preparation step wherein the Cannabis is ground into particles. The grinding step is a critical step that must be executed properly to achieve the preferred objects of the invention.

Grinding risks degradation of the product by generation of heat, by clumping of sticky materials, and by loss of material to the grinding instrument. All aspects must be carefully controlled to achieve superior results.

Preferably, Cannabis will be ground to sieve through a mesh of not larger than about 0.1 mm to about 3 mm, or any 0.1 mm increment therebetween, more preferably not larger than about 1.5, mm in any surface dimension. In one embodiment, dried Cannabis material is obtained for use in the grinding step. Cannabis material may include, without limitation, the leaves, inflorescences, flowers, or buds of one or more Cannabis plants. The grinding step may use any grinding method or methods, such as hand grinding, machine grinding, or use of a chipper or mulcher, provided that a consistent milled size product as homogenous as possible is generated without degradation. Degradation can occur through generation of heat during the grinding process and should be carefully controlled.

In another embodiment, the grinding step may grind the material to a particle size wherein 85-95% of the mass of particles have a maximum length less than about 0.1 mm to about 3 mm, or any 0.1 mm increment therebetween, preferably not larger than about 1.5 mm, and 5-15%, or any percentage increment therebetween, of the mass of the material are in particles have a length greater than about 0.1 mm to about 3 mm, or any 0.1 mm increment therebetween, preferably not larger than about 1.5 mm. Initial grinding may be followed by one or more filtering or sieving stages, for example to filter out stems or sticks. An illustrative mesh size for filtering or sieving the ground plant material may have an aperture size in the range of about 0.25 mm to 1.5 mm in its longest surface dimension. A coarse grinder having aperture larger than 1.5 mm is not preferred because it leads to uneven temperature distribution during vaporization.

In some embodiments, the kief portion may be separated from the other plant material during the grinding step. In a preferred embodiment, the kief is not separated from the preparation used for encapsulation, or if it has been separated, it is added back in. In general, the kief portion will be a large portion (by mass) of the smaller material, generally in the 65-125 micron range.

In some embodiments, additives may be added to the pod. In some embodiments, the pods(s) include certain terpene or terpenoid compounds. For example, in some embodiments, pods include added limonene, providing a lemon scent to the consumer. In other embodiments, pods include added myrcene. Such pods including added myrcene may be useful as sleep aids.

It has been observed that milled or ground material demonstrates improved flowability, and therefore is more suitable for use in preparing pods in accordance with the current invention. In general, the finer the grind, the better degree of flowability demonstrated by the material.

It has also been surprisingly observed that the content of ground or milled Cannabis preparations of Cannabis plant material contain lower proportions of cannabinoids than corresponding unground/unmilled plant material. It has also been surprisingly observed that the fraction of material that passes through a sieve, for example having a 30, 60, or 120 mesh, contains an elevated proportion of cannabinoids (e.g. THC) than does the input material. Hence, it has been surprisingly observed that sieving or meshing ground/milled Cannabis plant material in accordance with the present invention has the effect of cheaply and easily, without any chemical processing, increasing cannabinoid concentration in pure, additive free, Cannabis plant material (in the pass through fraction) and reducing concentration (in the retained fraction). It has also been surprisingly observed that by selecting an appropriate mesh size, one can selectively control the degree of concentration increase (in the pass through fraction) or decrease (in the retained fraction).

In a preferred embodiment, the Cannabis pod is prepared by obtaining or preparing a ground or milled Cannabis preparation. The milling may be performed by any suitable means, for example a household grinder or miller such as the Magic Bullet. The ground or milled preparation is then passed through a mesh or sieve. A fraction of material will then be retained on the sieve or mesh, while another fraction will pass through. The size of the particles which pass through will vary depending on the size of the openings in the mesh or sieve.

The concentration of one or more cannabinoids of interest (e.g. THC) is then measured in one or both of the pass-through and retained fractions. Based on the measured concentration, a target amount of retained fraction material, pass-through material, or a combination thereof, to be incorporated into the Cannabis pod is determined, such that the pod includes a desired defined dose of the one or more of one or more cannabinoids of interest. Finally, the desired amount of the material is placed into suitable container/material to form the pod.

### Dose Selection

The defined dose Cannabis pods of the present invention have defined doses of one or more physiologically active compounds from Cannabis. In some embodiments, the pods of the present invention have defined doses of one or more cannabinoids. Preferred defined dosages apply to cannabinoid compounds including, but not limited to: THC, THCA, CBD and CBDA. In some embodiments, the pods may comprise a defined dose selected from the following ranges: about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THCA THCA, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the pods comprise about 0 mg, 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg of one of the foregoing compounds. In some embodiments, the pods of the present invention have defined dosages for more than one of the foregoing compounds. For example, in some embodiments, the pods comprise from about 0 mg-1 mg, or any 0.1 mg interval therebetween THC, about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the pods comprise from about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBDA. In some embodiments, the compositions are substantially free of THC-type cannabinoid compounds. For example, in some embodiments the pods comprise from about 0 mg-1 mg, or any 0.1 mg interval therebetween THC, 0 mg-1 mg THCA, or any 0.1 mg interval therebetween, about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD, and about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg CBD.

In some embodiments, the pods described herein comprise an “effective” amount of one or more of the cannabinoid ingredients described herein. The term “effective amount” refers to an amount of the one or more cannabinoid ingredients sufficient to induce a change in an individual user. An effective amount also means an amount of the one or more cannabinoid ingredients that is needed to provide a desired level of cannabinoid(s) in the bloodstream of an individual user to provide an anticipated physiological response. An effective amount of a cannabinoid ingredient can be administered in one administration, or through multiple administrations of an amount that total an effective amount, preferably within a 24-hour period. It is understood that the effective amount can be the result of empirical and/or individualized (case-by-case) determination on the part of the individual user. For example, a therapeutically effective amount of said one or more cannabinoid ingredients may be in the range of about 1 mg to 2,000 mg, or any 1 mg or 10 mg interval therebetween total cannabinoids per day.

In some embodiments, an effective amount of said one or more cannabinoid ingredients may be in the range of about 1 mg-5 mg, or any 1 mg or 0.1 mg interval therebetween per day. For example, for an adult, about 1-2 mg, or 0.1 mg interval therebetween, a day total cannabinoids may provide a very low end dose below the psychoactive threshold.

In some embodiments, an effective amount of THC may be in the range of about 5 mg-90 mg, or any 1 mg interval therebetween. For example, most vapers inhale about 10 to 30 mg of THC to establish a mild, temporary, psychoactive effect.

In some embodiments, a composition of the present invention may comprise THCA in an amount between 5-165 mg, THC in an amount less than 1.0 mg, and CBDA in an amount between 0.1-70 mg, and have a total mass of 100-500 mg.

In some embodiments, a composition of the present invention may comprise THCA in an amount less than 5.0 mg, THC in an amount between 5-135 mg, and CBD in an amount between 0.1-70 mg, and have a total mass of 100-500 mg.

In some embodiments, a composition of the present invention may comprise THCA in an amount less than 1.0 mg, THC in an amount less than 1.0 mg, and CBD in an amount between 5-90 mg, and have a total mass of 100-500 mg.

In some embodiments, an effective amount of CBD for treating disorders such as pain, nausea, chronic pain conditions may be in the range of about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg per day. Preferably, the amount of CBD may be about 50 mg per day. For example, a recommended CBD serving standard may be about 25 mg of CBD taken twice a day.

In some embodiments, an effective amount of THCA may be in the range of about 0 mg, about 9 mg, about 90 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between 0 mg and 100 mg.

The therapeutic effects induced in an individual can be somewhat predictable but may vary from one individual to the next. The precise amount of cannabinoids required to induce an effect in an individual will depend upon numerous factors, e.g. type of cannabinoid(s), activity of a composition, intended use (e.g. number of doses per day), individual user considerations, methods of consumption, and others, which can readily be determined by one skilled in the art.

An achievement of the invention is that by using the composition of the invention, users and medical advisors for the first time have knowledge of the exact doses they are employing.

### Analytical Testing to Establish the Defined Dose

Two key analytical steps for the invention include:
1. Determining amounts of cannabinoids, especially THCA, THC, CBDA, and CBD of the Cannabis preparation prior to encapsulation of an individual pod; and
2. Determining amounts of cannabinoids, especially THCA, THC, CBDA, and CBD after encapsulation in the final Cannabis pod product.

For either step, any chemical analytical method may be employed to determine the amount of the cannabinoids. Many methods are available to those skilled in the art, such as those found in Thomas, B F and El Sohly, M 2015 “The Analytical Chemistry of Cannabis: Quality Assessment, Assurance, and Regulation of Medicinal Marijuana and Cannabinoid Preparations” (Elsevier). See also Wang et al. (2016) Decarboxylation Study of Acidic Cannabinoids: A Novel Approach Using Ultra-High-Performance Supercritical Fluid Chromatography/Photodiode Array-Mass Spectrometry. Cannabis Cannabinoid Res.; 1(1): 262-271; and Wang et al. (2017) Quantitative Determination of Cannabinoids in Cannabis and Cannabis Products Using Ultra-High-Performance Supercritical Fluid Chromatography and Diode Array/Mass Spectrometric Detection. J Forensic Sci.; 62(3):602-611). A particularly recommended approach is found at Mudge et al. (2017) Anal Bioanal Chem (2017) 409:3153-3163 DOI 10.1007/s00216-017-0256-3.

The two key analytical steps are applied in a similar but distinct fashion in the process.

For example, prior to encapsulation, the Cannabis preparation must contain the desired amounts of the selected cannabinoids to result in a pod having the desired defined dose. Due to the unpredictability of cannabinoids in the Cannabis plant that may result from strain variety, cultivation and harvesting conditions, the amount of cannabinoids per unit mass is never guaranteed. Nor are the relative ratios of cannabinoids. The first analytical step therefore will be to harvest and grind the “process batch” and to perform a batch assay to ascertain amounts of all relevant cannabinoids in a representative sample. The representative sample and measurement technique must be sufficient to represent all samples of the process batch within the degree of variability tolerated by the overall process, which as described below is +/−5% of the defined dose of each cannabinoid.

Having identified the cannabinoid amounts of the process batch, it will need to be adjusted with filler to obtain the desired final amounts of cannabinoids for the preparation. In the method of the invention, the “filler” will be selected from among ground Cannabis preparations having known cannabinoid amounts which are different from the process batch. The operator will have available a series of “filler batches” with relatively higher or lower amounts and ratios of cannabinoids. The batch assay will allow the operator to identify by simple algorithm which amount of which filler batch(es) are required to obtain the final defined dose for the preparation before encapsulation.

An alternative method to adjust the cannabinoid amounts of the process batch is to add cannabinoid concentrates (having predominantly aqueous carriers) and/or cannabinoid oils (predominantly lipid carriers). The operator will have available a variety of cannabinoid concentrates and/or oils of known cannabinoid concentrations (such as may be prepared by those skilled in the art). Again, by simple algorithm the operator can determine which amount of which concentrate or oil is required to bring the process batch into conformity with the desired dose of the final product. The concentrate or oil is contained in a liquid volume which, when mixed with the process batch, is fully absorbed by the dried Cannabis and does not cause clumping or sticking prior to encapsulation. Such a composition may be considered an enriched Cannabis product.

After the filler batches are selected, they will be added, combined, and mixed thoroughly with the process batch. The final encapsulation ready preparation may also be batch assayed to ensure the defined dose cannabinoids are present in the proper mass. Any final preparation which is not within tolerance levels is discarded or re-processed until desired cannabinoid levels are obtained. The final tolerance level is within 5%, preferably within 2% and most preferably within 1% of the desired in-going amount of each cannabinoid in the preparation.

The second analytical step is performed after the encapsulation has been completed. It may be performed before or after the packaging and labelling process. In the second analytical step, a representative sample of defined dose pods are analyzed to confirm the amount and ratios of cannabinoids present. This quality control step confirms whether the temperature and UV exposure conditions of the process have been properly observed. The chemical analytical tools employed may be the same or different from those employed in the first analytical step. It may be presumed that the contents of the pod are released from the encapsulating textile prior to analysis.

It is also noted that since moisture content is relevant to the flowability and stickiness of the ground Cannabis preparation prior to encapsulation, the process operator will sometimes seek to determine and possibly adjust its moisture content. As the skilled person in the art will be aware, the moisture content of a Cannabis preparation may be obtained by Karl Fischer titration, following extraction of the vaporisable material in a suitable solvent such as methanol, or by heating the vaporisable material in a Stromboli sample oven. The Karl Fischer titration method is described in Fischer, K., Angew. Chem. (1935) 48 (26): 394-396.

### Encapsulation and Sealing Step

In one embodiment, the invention provides a method of producing a Cannabis pod by encapsulating loose plant source material within a textile of primarily natural fiber. In various embodiments, the textile comprises materials that exhibit high temperature tolerance and are able to withstand relatively high heat temperatures. In one embodiment, the textile is durable at high temperatures and displays low inspiratory resistance. In one embodiment, the textile may have a basis weight of between about 12-28 g/m². In a preferred embodiment, the textile is durable up to about 230° C. and displays an inspiratory resistance of less than about 74.5 Pa/litre(air)/sec.

In some embodiments, a textile of primarily natural fiber means the underlying fiber is extracted from plants or woody source material. It may be pure or semi-synthetic as long as the final material contains at least 50% natural fiber. Further, the textile desirably comprises a fibrous material that is primarily naturally sourced. Examples of naturally sourced materials for use with this invention include without limitation, hemp, Cannabis material, jute, flax, coir, cotton, kenaf, acetate, soybean fibre, paper-based materials such as baking parchment, hemp paper, and cardboard, or any combination thereof. In a preferred embodiment, the fibrous material may be of any suitable material which would permit air to be drawn into the interior region of the pod, and a vaporized aerosol to be drawn therefrom. Light-weight materials in the range of 12-28 g/m², similar to cigarette paper, are preferred, as long as they are strong enough to prevent tearing during typical manipulation during production and use by the user. For reference, ordinary tea bags typically have a basis weight of 14-18 g/m². Paper towel typically has a basis weight of 20-24 g/m².

The textile preferably adds a minimum of resistance to air-flow passing through it. In some embodiments, the textile mesh creates minimal inspiratory resistance similar to face-masks worn during surgery or in urban settings. Such masks demonstrate an inspiratory resistance of less than about 74.5 Pa/litre(air)/sec, sometimes stated as less than about 5.0 mm H₂O/cm² or less than 50 Pa/cm² inspiratory resistance. Low resistance permits the rapid heating and vaporization of cannabinoids inside the pod, while requiring a minimum of excess inspiration effort by the consumer. See Kim J H et al. (2016) Pressure Drop of Filtering Facepiece respirators. Int J Occup Med Environ Health. 2015; 28(1): 71-80. doi: 10.13075/ijomeh.1896.00153. The textile of primarily natural fiber will therefore provide minimal inspiratory resistance similar to a face mask, e.g. less than 50 Pa/cm².

High-temperature tolerance is to ensure the pod remains intact during the vape process. Vape proceeds at a temperature below the auto-ignition point of dried Cannabis. The auto-ignition point of dried Cannabis corresponds to the auto-ignition point of many papers at about 232° C. (451° F.). Vaping therefore typically proceeds below 232° C. (often 226° C. is selected). Lower temperatures may also be used e.g. 150-185° C., providing different selections of cannabinoids and terpenoids in the vapor. The textile is selected to ensure its integrity is retained at all normal vape temperatures.

The textile is also selected to ensure that it carries negligible chemical compounds which will evaporate or join the out-flow stream of vapor passing through the pod. Many types of paper carry residual additives which will evaporate in the 150-226° C. range. Such papers are preferably avoided. Hemp paper, preferably produced from organically grown hemp, is a preferred textile in the invention, due to its similar species origin as the internal plant material. A preferred embodiment avoids the use of synthetic polymers or polymers with contaminants or additives that would be released under vaping conditions.

In some embodiments, the textile is integrally formed as a single unitary body. The edges of the unitary body may be joined at a junction to seal the Cannabis material therein. In some embodiments, more than one textile body may be used. When more than one textile body is used, their edges may be connected to encapsulate the Cannabis material therein.

In some embodiments, the junction or connection of one or more textile bodies includes the use of an adhesive material. Preferably, the adhesive material is a non-chemical adhesive. In some embodiments, the method of joining or connecting the edges of one or more textile bodies does not include external adhesive and is achieved with heat-sealing paper or organic thread stitching.

In some embodiments, the textile surrounds the ground Cannabis on substantially all sides. The textile may substantially seal the ground Cannabis, and may therefore be used to store, distribute and/or sell individual prepackaged portions of Cannabis material or to maintain freshness. The pod enables individual portions of Cannabis material to be easily placed within a vaporizer or similar device, which in some embodiments may be a teapot or similar device.

Critical temperatures for selecting process temperature control may be identified from the following list:

| Process | Temperature |
|---|---|
| Accelerated dehydration (drying) | Up to 95° C. |
| THCA decarboxylation to THC | 105° C. or higher |
| Curing of Cannabis | 105-113° C. for 30-45 |
| flower (baking) | minutes or longer |
| Evaporation of | 126° C. |
| Aromatic terpenoids | |
| THC | 157° C. |
| CBD | 160-180° C. |
| CBN | 185° C. |
| Efficiency of cannabinoid | |
| evaporation during vaporization | |
| “about half efficiency” | 150-180° C. |
| “highest efficiency” | 226° C. |
| Combustion/Auto-ignition | About 232° C. and higher |
| temperature of dried Cannabis | |
| Smoking of Cannabis | 232-1200° C. |

### Pod Mass and Shape, and Textile Fringe/Edge

The pods may be oval, spherical, cylindrical, conical, cubic, rectangular, square, pyramid and the like. The pods may be envelope-like or bag-like. The pods may be of any other suitable or desirable shape. It should be appreciated that a number of different types, shapes, sizes of pods may be possible to accommodate a device, such as a vaporizer, a pipe, a bong, a “oneie,” and the like. The finished total mass of the Cannabis pods of the invention ranges from about 0.1 g to about 0.5 g.

In some embodiments, the pod is shaped and sized to releasably and snugly engage the vaporising chamber of a vaporizer device. In one embodiment, the pod substantially defines the vaporising chamber of a vaporizer device to facilitate heat transfer from the vaporising chamber to the pod. Advantageously, the pod is adapted to releasably engage the vaporising chamber of a vaporizer device.

It will be appreciated that the amount of plant source material and the dimensions of the pod are not meant to be limiting. Pod sizes cover a range of options useful for a private single low dose user, to use of a single pod by a larger group of individuals and/or at higher doses. For example, the diameter of the pod may be about 5 mm to 20 mm, or larger, and the thickness may be about 1 mm to 10 mm, or larger. The pod may be wider or narrower, or thinner or thicker as may be desired.

The edges of each pod can be shaped to provide greater surface area to enhance heat transfer. For example, pods may have indentations or ridges along the outer side of each pod, or concave surfaces, so as to further increase the surface area of the pod or to enhance air flow into the pod.

Pods may be embossed with a signifier such as a logo or design or shape-feature on at least one side of the pod. Signifiers include a barcode, or another type of machine readable code to identify the particular type of pod. Embossing of the pod with a signifier may take place contemporaneously with, or subsequent to, encapsulation.

Preferred shapes are those designed to fit in the bowls of leading vaporizer devices such as the Firefly 2 (thefirefly.com), Crafty (Storz & Bickel), Mighty (Storz & Bickel), or others including the DaVinci IQ, PAX 3, Arizer Solo 2 & Air 2, Hydrology9 by Cloudious9, Ghost MV1, Atmos VICOD 5G, and the Atmos Jump. Additional popular vaporizers for which pods of the invention may be designed include the Airvape XS (Apollo), Arizer Air II, Arizer Extreme Q, Arizer Go ‘ArGo’ (all from Arizer), G-Pen Elite, G-Pen Pro and G-Pro (all from Grenco Science), the Boundless CF, CFV or CFX (Boundless), Ascent (DaVinci), Haze V3.0 (Haze Tech), Volcano Classic, Volcano Digital and the Volcano Plenty (Storz and Bickel).

### Defined Dose Cannabis Pod with Pull-Tab and Tearable Strip

In some embodiments, the pod may include further structure such as coupling features (e.g., threaded portions, elastomeric covers, pull-tabs, tearable strips, flaps etc.) for attaching to the pod. For example, when suitably coupled, the coupling feature may form a seal with the fibrous material of the pod. Such a seal may be appropriate for locking in the overall aroma and freshness of the Cannabis composition, preserving the contents therein. The coupling feature may also function to block external light and air, while the pod is sealed.

The coupling feature may be configured to facilitate opening of the pod, facilitating pouring or dispensing of the ground Cannabis material from the pod into a vaporizer device, pipe, cigarette or similar vaporization or combustion device.

In some embodiments, the coupling feature may be temporarily joined to the pod across one or more openings with an adhesive, enabling a person to easily peel the coupling feature from the pod. In other embodiments, the coupling feature may be secured to the pod in a more permanent fashion across one or more openings, wherein the coupling feature is torn away from remaining portions of the pod so as to expose said opening(s). In one embodiment, the coupling feature may be torn away and additional openings may be manually punctured (with a sharp object).

In one embodiment, the pod comprises the defined dose of Cannabis contained between two separate textile sheets. The sheets are held together by a thread around the outer edge of the pod which sews the two sheets together. The thread is selected to be durable up to at least 230° C. The thread may be configured to be fixed in place, alternatively it may be releasable by the user so as to release the inner Cannabis contents of the pod.

In another embodiment, the pod comprises a single textile sheet. In this case a draw string sewn into the outer edge of the sheet is employed to draw the single sheet closed as a purse. The Cannabis is contained in the body of the purse so formed. The draw string may be configured to be permanent or to be releasable by the user. The draw string also conveniently may be tagged at the end, or somewhere along its length, to provide a surface area on which can be placed a signifier which indicates the defined dose of the pod (as further described below).

In one embodiment, the coupling feature is a pull-tab. The pull-tab is configured to be torn or peeled away so as to open the pod. The pull-tab may be affixed to a stiffening structure, wherein the stiffening structure comprises embossed, corrugated, creased, folded or heat-shaped material. The stiffening structure may be embossed in or underlying said textile and arranged in a pattern such that pulling of the pull-tab by a user tears the textile along a creased or weakened folding line that defines the intended tearing line across the textile. In one embodiment, the intended folding line may extend in a direction substantially parallel to the long axis of the pod. In one embodiment, a plurality of elongated stiffening elements extends from the pull-tab towards outer edges of the pod.

### Additives

In certain embodiments, the ground Cannabis preparation used for encapsulation will contain non-Cannabis additives. A wide range of additives may be employed. Some additives may be useful as stabilizers or binders for the pod. Others may be humectants, vaporization aids, and fillers of all kinds.

A humectant is a hygroscopic substance that has an affinity to form hydrogen bonds with molecules of water and is used to produce a visible exhaled aerosol (i.e. vapour) when the product is in use. Suitable humectants for inclusion in a vaporisable material according to the present invention include propylene glycol, also known as 1,2-propanediol or propane-1,2-diol and having the formula C3H8O2 or HO—CH2-CHOH—CH3, and glycerol, also known as glycerine and having the formula C3H8O3. In a preferred embodiment, the humectant is propylene glycol. The present invention provides a Cannabis preparation having a moisture content of from about 3 to 5 wt %, and further comprising a humectant in an amount of at least 20 wt %.

A wide variety of flavouring agents, scents, perfumes and colouring agents, in addition to terpene additives such as limonene and myrcene, may also be employed in certain embodiments.

### Packaging Step

After the pod is encapsulated and sealed, delivery to market is achieved by

a. packaging the pod individually in a blister pack impermeable to gas exchange; or

b. packaging the pod in a re-sealable multi-pod package impermeable to gas exchange.

The Cannabis pods are preferably provided in a sealed package, which functions as an absolute barrier enabling the moisture content and flavour to be retained over time. The term “sealed package” refers to a gas-impermeable container having a hermetic closure which in the context of the present invention includes a blister pack. The Cannabis pods may be individually sealed and packaged in blister packs. The blister packs may be designed to be child resistant and/or senior friendly in order to increase safety and convenience. While physically protecting the pods, the blister pack controls humidity and is impermeable to gas exchange thereby maintaining freshness and enhancing the shelf life of the pods.

Examples of the substantially gas exchange impermeable packaging include, but are not limited to, Al/Al blister, and Al-polychloro-3-fluoroethylene homopolymer/PVC laminate blister. Alternatively, the sealed package may be a re-sealable multi-pod package impermeable to gas exchange. Such packages may be adapted from those known in the art by those skilled in the art to accommodate Cannabis pods of the invention. Ideally, the encapsulated and sealed pod should be processed and packaged as quickly as possible to ensure that moisture is not lost from absorbed into the composition.

Optionally, the packaging material is selected from materials which block some or all transmission of ultraviolet radiation. Use of such material will delay or prevent decarboxylation of cannabinoids such as THCA and CBDA, thereby preserving the defined dose characteristics of pod of the invention during storage and shelf exposure.

Pods are expelled from production into the open blister cavities. Cavity depth and shape must be suitable for the pod. The open blister cavity is then sealed with a gas impermeable membrane to maintain quality of product and to reduce dehydration, rehydration or oxidation. To eliminate oxidation altogether, the packaging may be performed in an inert gas atmosphere. Optionally the blister is packed in an inert gas atmosphere such as nitrogen gas comprising little or no oxygen. To achieve this objective, the final sealing step of the packaging method may be operated in the inert gas atmosphere in a gas enclosure protected from ambient air.

### Association with Signifier

The method and composition of the invention associates the Cannabis pod with a signifier which allows the consumer to determine the defined dose of selected cannabinoids therein. A “signifier” means a visual mark or symbol that the consumer recognizes as referring to a specific defined dose. The signifier chosen may have elements of meaning, such as a number and unit, (e.g. “5 mg” or “10 mg” or simply “5” or “10”) or it may be an abstract signifier, where its meaning, in terms of defined dose, can be determined by reference to a standard. The meaning may be determined directly by the consumer or indirectly via a device.

The signifier may be associated directly with the Cannabis pod after encapsulation by such means as embossing, or by colour, pattern or shape feature. Alternatively, the signifier may be associated with the packaging. The packaging may include signifiers directly interpreted by consumers or signifiers which are machine readable codes. In all embodiments, the signifier allows the consumer to determine the defined dose of selected cannabinoids therein.

### Example 1—Cannabis Preparation: Milling and Sieving

Milling and sieving may be employed to develop a finer grain of product for pod manufacturing. Surprisingly, as is shown below, milling and sieving may lead to increased potency of Cannabis in the finer portions of the ground product.

Mesh sizes referred to herein are standard US mesh size designations. The mesh number corresponds to number of openings in one square inch of screen, e.g. a 100-mesh screen has 100 openings. As the mesh size increases, the size of the particles decreases. Higher numbers equal finer material. A 60 mesh sieve has a mesh opening of 250 μm. With a 60 mesh sieve, milled as indicated, 66.5% by mass is retained on the sieve and 33.5% by mass passes through onto the sieve pan.

### Flower Milling

Approximately 10 g of dried flower was placed into a mill and milled for 1-3 minutes to finer powder. The milled flower was then pushed through a) 30 mesh sieve with a 595 μm opening b) 60 mesh sieve with a 250 μm opening and c) 120 mesh sieve 125 μm opening. The powder from the Pink Kush has a light green colour to it. The sieved material collected on the mesh and material collected in the sieve is suitable for use in manufacturing of pods in accordance with the present invention.

| | Total | Sieve | Amount | % |
|---|---|---|---|---|
| Formulation | (g) | No. | Retain (g) | Retain |
| (THCA) | 9.69 | 30 | 1.81 | 18.7 |
| | | 30- | 7.88 | 80.5 |
| | | Sieve Pan | | |
| | 11.98 | 60 | 7.1 | 59.2 |
| | | 60- | 4.88 | 40.7 |
| | | Sieve Pan | | |
| | 10.55 | 120 | 7.03 | 66.6 |
| | | 120- | 3.52 | 33.3 |
| | | Sieve Pan | | |

S = Sieved material collected on pan
US = Unsieved material retained on mesh

After milling and putting the milled material through 30 mesh, approximately 19% retained on 30 mesh sieve, 81% passed through the 30 mesh sieve. On the 60 mesh sieve 59% of the flower material was retained and 41% passed through the 60 mesh sieve. With the 120 mesh sieve approximately 67% was retained on the 120 mesh sieve and 33.3% passed through the 120 mesh sieve.

### Example 2—Potency Calculation for an Exemplary 56 mg THCA Pod

To manufacture 56 mg THCA flower using Pink Kush the calculation is as follows:

$$Potency of THCA = 23.34 \% \\ \begin{matrix}Pod Weight for 56 mg THCA = Defined Dose ( mg ) / Potency of THCA \\ = 56 mg / 0.2334 \\ = 239 mg pod weight\end{matrix}$$

The composition and methods described herein are illustrative and not limiting on the claims of the invention more particularly set out below.

1. A method of preparing a Cannabis pod comprising a desired defined dose of one or more desired cannabinoids, the method comprising:
(a) preparing or obtaining a ground or milled Cannabis plant material preparation;
(b) passing the Cannabis plant material preparation through a mesh or sieve to obtain a retained fraction of the material which is retained in the mesh or sieve and a pass-through fraction of the material which has passed through the mesh or sieve;
(c) determining the content of the one or more cannabinoids of interest in the retained fraction and/or the pass-through fraction;
(d) based on the content determined in step (c), determining a target amount of retained fraction material, pass-through material, or a combination thereof, to be incorporated into the Cannabis pod such that the pod includes the desired defined dose of the one or more desired cannabinoids; and
(e) encapsulating the target amount within a textile, preferably of primarily natural fiber, permitting air flow at or below minimal inspiratory resistance,
thereby forming the Cannabis pod.

2. A defined dose Cannabis pod comprising a defined dose of:
(a) 0.1-165 mg tetrahydrocannabinolic acid (THCA),
(b) 0.1-135 mg tetrahydrocannabinol THC,
(c) 0.1-100 mg cannabidiolic acid CBDA; and/or
(d) 0.1-100 mg cannabidiol CBD
in a ground Cannabis plant material preparation contained within a textile of primarily natural fiber, and permitting air flow at or below minimal inspiratory resistance.

3. The pod of claim 2 wherein the Cannabis plant material preparation includes material derived from one or more Cannabis plants from a Cannabis species selected from the group consisting of Cannabis sativa, Cannabis indica, Cannabis ruderalis, and hybrids thereof.

4. (canceled)

5. The pod of claim 2 wherein the Cannabis plant material preparation includes material prepared from Cannabis inflorescence.

6. The pod of claim 2 further comprising one or more additives selected from among terpenes, terpenoids, pod stabilizers, humectants, vaporization aids, fillers and flavours.

7. The pod of claim 2 wherein the amount of THC is less than a psychotropic dose.

8. The pod of claim 2 wherein the amount of THCA is less than 1.0 mg.

9. The pod of claim 2 comprising:
THCA in an amount between 1-165 mg,
THC in an amount less than 1.0 mg; and
CBDA in an amount between 0.1-70 mg,
and having total mass 100-500 mg.

10. The pod of claim 2 comprising:
THCA in an amount less than 5.0 mg,
THC in an amount between 1-135 mg; and
CBD in an amount between 0.1-70 mg,
and having total mass 100-500 mg.

11. The pod of claim 2 comprising:
THCA in an amount less than 1.0 mg,
THC in an amount less than 1.0 mg; and
CBD in an amount between 5-90 mg,
and having total mass 100-500 mg.

12. The pod of claim 2 wherein one or more of the Cannabis plant material preparation is prepared from one or more Cannabis plants selected from the group consisting of Charlotte's Web and other high CBDA, low THCA plant varieties.

13. The pod of claim 2 wherein the textile contains no synthetic polymer.

14. The pod of claim 2 wherein the textile is organically sourced.

15. The pod of claim 2 wherein the pod comprises no chemical adhesive.

16. The pod of claim 2, wherein the pod is sealed individually in a blister pack impermeable to gas exchange, and optionally in an inert gas atmosphere.

17. The pod of claim 2, wherein the pod is in a re-sealable multi-pod package impermeable to gas exchange.

18. The pod of claim 2, wherein the pod or a packaging is associated with a signifier which provides an observer with information on the defined dose of selected cannabinoids in the composition.

19. The pod of claim 2 or 18 wherein the pod or the packaging comprises a pull-tab disposed to release the pod contents at the user's option, said pull-tab affixed to a stiffening structure embossed in or underlying said textile and arranged in a pattern such that pulling of the pull-tab by a user tears the textile along a creased or weakened folding line that defines the intended tearing line across the textile, and wherein the intended tearing line extends in a direction substantially parallel to the long axis of the pod.

20. The pod of claim 19, wherein the stiffening structure comprises embossed, corrugated, creased, folded or heat-shaped material, and wherein a plurality of elongated stiffening elements extends from the pull-tab towards outer edges of the pod.

21. The pod of claim 19, wherein the pull-tab includes a concave groove dimensioned to aid in grasping thereof.

22. The pod of claim 19, wherein the packaging is associated with a signifier which provides an observer with information on the defined dose of THC in the composition.

23. The pod of claim 19, wherein the packaging is associated with a signifier which provides an observer with information on the defined dose of cannabinoids in the composition, wherein such cannabinoids are selected from among THCA, THC, CBDA, and CBD.

24. A method of delivering a cannabinoid to a subject, the method comprising:
(a) providing a pod comprising a Cannabis preparation comprising a defined dose of the cannabinoid, wherein the pod is coupled to a combustion device;
(b) igniting the combustion device, wherein the igniting causes the Cannabis preparation contained in the pod to reach a temperature sufficient to produce a vapor comprising the cannabinoid; and
(c) inhaling the vapor, wherein the inhaling is performed by the subject, thereby delivering the cannabinoid to the subject.

25. The method of claim 24, wherein the defined dose comprises
(d) 0.1-165 mg tetrahydrocannabinolic acid (THCA),
(e) 0.1-135 mg tetrahydrocannabinol THC,
(f) 0.1-100 mg cannabidiolic acid CBDA; and/or
(g) 0.1-100 mg cannabidiol CBD
in a ground Cannabis plant material preparation contained within a textile of primarily natural fiber, and permitting air flow at or below minimal inspiratory resistance.

26. The method of claim 25 wherein the Cannabis plant material preparation includes material derived from one or more Cannabis plants from a Cannabis species selected from the group consisting of Cannabis sativa, Cannabis indica, Cannabis ruderalis, and hybrids thereof.

27. The method of claim 25 wherein the Cannabis plant material preparation includes material prepared from Cannabis inflorescence.

28. The method of claim 25 further comprising one or more additives selected from among terpenes, terpenoids, pod stabilizers, humectants, vaporization aids, fillers and flavours.

29. The method of claim 25 wherein the amount of THC is less than a psychotropic dose.

30. The method of claim 25 wherein the amount of THCA is less than 1.0 mg.

31. The method of claim 25 comprising:
THCA in an amount between 1-165 mg,
THC in an amount less than 1.0 mg; and
CBDA in an amount between 0.1-70 mg,
and having total mass 100-500 mg.

32. The method of claim 25 comprising:
THCA in an amount less than 5.0 mg,
THC in an amount between 1-135 mg; and
CBD in an amount between 0.1-70 mg,
and having total mass 100-500 mg.

33. The method of claim 25 comprising:
THCA in an amount less than 1.0 mg,
THC in an amount less than 1.0 mg; and
CBD in an amount between 5-90 mg,
and having total mass 100-500 mg.

34. The method of claim 25 wherein one or more of the Cannabis plant material preparation is prepared from one or more Cannabis plants selected from the group consisting of Charlotte's Web and other high CBDA, low THCA plant varieties.

35. The method of claim 25 wherein the textile contains no synthetic polymer.

36. The method of claim 25 wherein the textile is organically sourced.

37. The method of claim 25 wherein the pod comprises no chemical adhesive.

38. The method of claim 25, wherein the pod is sealed individually in a blister pack impermeable to gas exchange, and optionally in an inert gas atmosphere.

39. The method of claim 25, wherein the pod is in a re-sealable multi-pod package impermeable to gas exchange.

40. The method of claim 25, wherein the pod or a packaging is associated with a signifier which provides an observer with information on the defined dose of selected cannabinoids in the composition.

41. The method of claim 25 or 40 wherein the pod or the packaging comprises a pull-tab disposed to release the pod contents at the user's option, said pull-tab affixed to a stiffening structure embossed in or underlying said textile and arranged in a pattern such that pulling of the pull-tab by a user tears the textile along a creased or weakened folding line that defines the intended tearing line across the textile, and wherein the intended tearing line extends in a direction substantially parallel to the long axis of the pod.

42. The method of claim 41, wherein the stiffening structure comprises embossed, corrugated, creased, folded or heat-shaped material, and wherein a plurality of elongated stiffening elements extends from the pull-tab towards outer edges of the pod.

43. The method of claim 41, wherein the pull-tab includes a concave groove dimensioned to aid in grasping thereof.

44. The method of claim 41, wherein the packaging is associated with a signifier which provides an observer with information on the defined dose of THC in the composition.

45. The method of claim 41, wherein the packaging is associated with a signifier which provides an observer with information on the defined dose of cannabinoids in the composition, wherein such cannabinoids are selected from among THCA, THC, CBDA, and CBD.
